(19) United States
(12) Patent Application Publication
(10) Pub. No.: US 2026/0041634 A1
(43) Pub. Date: Feb. 12, 2026

(54) VISCOSITY REDUCING EXCIPIENTS AND COMBINATIONS THEREOF FOR HIGHLY CONCENTRATED NUCLEIC ACID COMPOSITIONS

(71) Applicant: MERCK PATENT GMBH, DARMSTADT (DE)

(72) Inventors: Tobias ROSENKRANZ, Darmstadt (DE); Tanja HENZLER, Darmstadt (DE); Andre KIESEWETTER, Darmstadt (DE)

(73) Assignee: MERCK PATENT GMBH, DARMSTADT (DE)

(21) Appl. No.: 19/100,416

(22) PCT Filed: Jul. 28, 2023
(86) PCT No.: PCT/EP2023/070990

§ 371 Date: Jan. 31, 2025

(30) Foreign Application Priority Data
Aug. 1, 2022 (EP) 22188168.3

(51) Int. Cl. A61K 9/08; A61K 9/19; A61K 31/7105; A61K 31/711; A61K 47/18; A61K 47/20; A61K 47/22; A61K 47/24; A61K 47/26; A61K 48/00

(52) U.S. Cl. CPC A61K 9/08; A61K 9/19; A61K 31/7105; A61K 31/711; A61K 47/183; A61K 47/20; A61K 47/22; A61K 47/24; A61K 47/26; A61K 48/00

(57) ABSTRACT

The present invention relates to liquid compositions comprising a nucleic acid having a reduced viscosity. Furthermore, the invention relates to methods for reducing the viscosity of a liquid compositions comprising a nucleic acid.

### TECHNICAL FIELD

The present invention relates to liquid compositions comprising a nucleic acid having a reduced viscosity. Furthermore, the invention relates to methods for reducing the viscosity of a liquid compositions comprising a nucleic acid.

### BACKGROUND

While for many decades nucleic acids themselves were solely subject of scientific investigations or served as a tool for genetic engineering work, today they are used as a therapeutically active substance or are an important starting material in the manufacturing process of novel active therapeutics including virus, gene therapy products and mRNA or DNA vaccines. With the recent success of novel treatment approaches, reflected in increasing number of medical approvals and positive results from clinical trials, the need for improved formulations for the administration of nucleic acid drugs and more efficient production methods has increased.

Aqueous solutions of high-molecular-weight DNA possess dynamic viscosities reaching hundreds and thousands of centi-poise (cP) even at such relatively low concentrations as several mg/ml (Creeth et al., J Chem Soc. 1947 September; 25:1141-5; Butler et al., Transactions of the Faraday Society, Volume 50, Pages 612-623195 1954). This makes the direct administration of naked therapeutic nucleic acids at common doses-from milligrams in DNA vaccines to tens of milligrams in gene therapy difficult because the viscosity of injectable pharmaceutical formulations should not exceed about 50 cP (Srinivasan et al., Pharm Res 30, 1749-1757, 2013) and ideally be less than half of that. Nucleic acid delivery systems like viral delivery vectors, cationic liposomes, and polycations, even if less viscous, often suffer from toxicity, low stability in vivo, and high cost (Dias and Lindman, DNA Interactions with Polymers and Surfactants, Wiley & Sons Inc. ISBN 978-0-470-25818-7; 2008 Saraswat et al., Indian J. Pharm. Sci., 71, pp. 488-498 2009)

Therefore, finding ways to reduce the viscosity of DNA solutions under pharmaceutically relevant conditions may be clinically useful both for formulating nucleic-acid drugs and as a stand-alone therapeutic. (Elkin et al. Intern. J. of Pharmaceutics Vol. 494, Iss. 1, 15, Pages 66-72; 2015) Similarly, the rise of novel technologies such as RNA based treatments and prophylactic vaccines (Particularly m-RNA)

Nucleic acids such as DNA, RNA, in particular m-RNA, can only be concentrated up to a few milligrams per milliliter. beyond that concentration level the viscosity of the solution would increase to an extent that set solution can no longer be injected into a patient or handled with standard lap of fill and finish equipment.

Nucleic acid therapeutics are usually administered parenterally, for example by intravenous (iv), intramuscular (im) or subcutaneous (sc) route. Subcutaneous injection is particularly popular for the delivery of nucleic acid therapeutics due to its potential to simplify patient administration (fast, low-volume injection) and reduce treatment costs (shorter medical assistance). To ensure patient compliance, it is desirable that subcutaneous injection dosage forms be isotonic and can be injected in small volumes (<2.0 ml per injection site).

At the same time, nucleic acid therapies usually requires milligram to tens of milligrams dosing. The combination of high therapeutic dose and low injection volume thus leads to a need for highly concentrated formulations of therapeutic nucleic acids. However nucleic acids possess a multitude of functional groups in addition to a potentially complex three-dimensional structure. This makes their formulation difficult, particularly when a high concentration is required.

One of the main problems is the high viscosity of solutions containing nucleic acids at concentrations above 1, 3 or 5 mg/ml. At such concentrations, nucleic acids tend to form highly viscous solutions largely due to non-native self-association. Another problem to be solved is the inability to increase the concentration of nucleic acids in a solution to concentrations above 1, 3, 5 or 10 mg/ml. Often, nucleic acid containing solutions cannot be handled by standard fill and finish or lab equipment. Yet another problem to be solved is that nucleic acid containing solution with high viscosities hamper processing steps which includes transfections of cell lines, polymerase chain reactions, and incorporation of nucleic acids into viral vectors.

The rheological properties of biomacromolecular solutions such as nucleic acid formulations not only complicates their parenteral administration in medical applications, but the high viscosity of nucleic acid solutions also presents a considerable challenge for their manufacture itself, and the intensification of the manufacturing process in particular.

Many known manufacturing methods of, in particular, relatively large amounts of nucleic acids like plasmid DNA or mRNA include filtration procedures, chromatographic purification steps or mixing, pump through or filling operations to process the product fluid stream. The performance of these steps is greatly impacted by the rheological behavior of fluid product stream to be processed. In the manufacturing process, highly concentrated nucleic acid formulations that are highly viscous present particular difficulties for ultrafiltration and sterile filtration. In addition, tangential flow filtration is often used for the buffer exchange and for the increase of nucleic acid concentration. However, because viscous solutions show an increased back pressure and shear stress during injection and filtration, the therapeutic nucleic acid is potentially destabilized and/or process times are prolonged. Said increased shear stress frequently results in a loss of product. Both aspects adversely affect process economics.

At the same time, high viscosity is unacceptable when it comes to administration as it significantly limits the injectability of the nucleic acid.

To solve these problems and/or to improve the stability of the solution, additives and excipients such as EDTA and sodium chloride are usually added in higher concentrations to biopharmaceutical formulations. However, the resulting solutions often cause pain due to high injection forces and resulting tissue damages. Some of these solutions may even be no longer administrable resulting in a lack of therapeutic options for the patient.

However, formulating nucleic acids like DNA or RNA, in particular mRNA, requires a careful selection of formulation additives and/or excipients to avoid denaturation and loss of biological activity. In addition, the excipients need to be pharmaceutically safe and physiologically compatible so to avoid any undesired side effects such as allergic reactions.

Consequently, the pharmaceutical industry has a strong need for additional pharmaceutically acceptable, viscosity-reducing excipients, especially as an alternative when standard solutions as mentioned above fail.

The problem to be solved is therefore the provision of excipients that can effectively reduce the viscosity of a nucleic acid solution. Furthermore, the problem to be solved is the provision of excipient combinations that can effectively reduce the viscosity of a nucleic acid solution.

During the bioprocess, the solutions have to be pumped through tubing and chromatography columns. At high viscosities, the flow rate through such columns is limited by said viscosity which leads to longer processing times, significant nucleic acid losses during chromatography or might lead to complete non-processability of the nucleic acid solution. Furthermore, when passing through a connector from the narrow tube into a less narrow column, shear forces may occur. Shear stress is a typical reason for nucleic acid to denature and potentially to aggregate and thereby reducing the yield of the process. Obviously, such shear stress induced aggregation has an adverse effect on process economics. Moreover, the gel bed within the chromatography column may be damaged by the high pressure.

Additionally, some nucleic acid are formulated into high concentration through tangential flow filtration (TFF). When the viscosity of the solution becomes critical, a gel-like layer may be formed near the membrane. Especially the membrane flux is significantly reduced yielding to an increased processing time and therefore to significant higher manufacturing costs. As discussed before, also during TFF shear stress may occur yielding to insoluble nucleic acid aggregates and a reduced yield.

Generally, it has been observed that highly viscous solutions develop a certain stickiness making it difficult to recover the complete solution from containers, out of tubing or to remove the entire substance from processing systems. This loss of substance leads to a significantly reduced product yield with the obvious adverse effect on process economics.

Furthermore, a problem to be solved is the provision of excipient combinations that can effectively reduce the viscosity of a nucleic acid solution.

High viscosity of nucleic acid solutions causes numerous difficulties in bioprocessing. Since known additives which hitherto are used for reducing the viscosity in corresponding nucleic acid solutions do not lead to sufficient viscosity-reducing effects in many cases, it is an object of the present invention to find new possibilities whereby corresponding viscosity-lowering effects can be improved and adverse effects on process economics can be reduced.

### SUMMARY OF THE INVENTION

The problem is solved by a liquid composition comprising a nucleic acid and at least one viscosity reducing excipient selected from the group consisting of arginine, phenylalanine, ornithine, meglumine, benzenesulfonic acid, pyridoxine and thiamine phosphoric acid ester. In a further embodiment the liquid composition comprises a nucleic acid and a combination of two excipients selected from the group consisting of arginine, phenylalanine, ornithine, meglumine, benzenesulfonic acid, pyridoxine and thiamine phosphoric acid ester. In a further embodiment the liquid composition comprises a nucleic acid and a combination of a first excipient selected from the group consisting of arginine, phenylalanine, ornithine and meglumine and a second excipient selected from the group consisting of benzenesulfonic acid, pyridoxine and thiamine phosphoric acid ester.

Likewise, the problem is solved by a method for reducing the viscosity of a liquid nucleic acid composition, comprising a step of adding at least one excipient selected from the group consisting of arginine, phenylalanine, ornithine, meglumine, benzenesulfonic acid, pyridoxine and thiamine phosphoric acid ester to the liquid nucleic acid composition. In a further embodiment a combination of two excipients selected from the group consisting of arginine, phenylalanine, ornithine, meglumine, benzenesulfonic acid, pyridoxine and thiamine phosphoric acid ester is added to the liquid nucleic acid composition. In a further embodiment a combination of a first excipient selected from the group consisting of arginine, phenylalanine, ornithine and meglumine and a second excipient selected from the group consisting of benzenesulfonic acid, pyridoxine and thiamine phosphoric acid ester is added to the liquid nucleic acid composition.

The use of at least two excipients allows to overcome the problem that many viscosity reducing excipients used at relevant concentrations can adversely affect nucleic acid stability by allowing for the use of a lower amount of each individual excipient and leveraging the stabilizing effect of a second excipient. Additionally, the use of at least two excipients leads to synergistic viscosity reduction.

Furthermore, the problem is solved by a lyophilized nucleic acid formulation of a composition comprising a nucleic acid and at least one viscosity reducing excipient selected from the group consisting of arginine, phenylalanine, ornithine, meglumine, benzenesulfonic acid, pyridoxine and thiamine phosphoric acid ester. In a further embodiment the composition comprises a nucleic acid and a combination of two excipients selected from the group consisting of arginine, phenylalanine, ornithine, meglumine, benzenesulfonic acid, pyridoxine and thiamine phosphoric acid ester. In a further embodiment the composition comprises a nucleic acid and a combination of a first excipient selected from the group consisting of arginine, phenylalanine, ornithine and meglumine and a second excipient selected from the group consisting of benzenesulfonic acid, pyridoxine and thiamine phosphoric acid ester.

Furthermore, the problem is solved by a kit comprising a composition comprising a nucleic acid and at least one first excipient selected from the group consisting of arginine, phenylalanine, ornithine, meglumine, benzenesulfonic acid, pyridoxine and thiamine phosphoric acid ester. In a further embodiment the composition comprises a nucleic acid and a combination of two excipients selected from the group consisting of arginine, phenylalanine, ornithine, meglumine, benzenesulfonic acid, pyridoxine and thiamine phosphoric acid ester. In a further embodiment the composition comprises a nucleic acid and a combination of a first excipient selected from the group consisting of arginine, phenylalanine, ornithine and meglumine and a second excipient selected from the group consisting of benzenesulfonic acid, pyridoxine and thiamine phosphoric acid ester.

An additional subject of the present invention is a method for reducing the viscosity of a liquid nucleic acid composition in a bioprocess, comprising the step of adding at least one excipient selected from the group consisting of arginine, phenylalanine, ornithine, meglumine, benzenesulfonic acid, pyridoxine and thiamine phosphoric acid ester to the liquid nucleic acid composition. In a further embodiment a combination of two excipients selected from the group consisting of arginine, phenylalanine, ornithine, meglumine, benzenesulfonic acid, pyridoxine and thiamine phosphoric acid ester is added to the liquid nucleic acid composition. In a further embodiment a combination of a first excipient selected from the group consisting of arginine, phenylalanine, ornithine and meglumine and a second excipient selected from the group consisting of benzenesulfonic acid, pyridoxine and thiamine phosphoric acid ester is added to the liquid nucleic acid composition.

### DETAILED DESCRIPTION OF THE INVENTION

The invention is directed to a liquid composition comprising a nucleic acid and at least one viscosity reducing excipient selected from the group consisting of arginine, phenylalanine, ornithine, meglumine, benzenesulfonic acid, pyridoxine and thiamine phosphoric acid ester.

A “nucleic acid” is herein defined as macromolecule composed of nucleotides. A “nucleotide” is a monomeric structure of the nucleic acid comprised of three components: a 5-carbon sugar, a phosphate group and a nitrogenous base. The two main classes of nucleic acids are deoxyribonucleic acid (DNA) and ribonucleic acid (RNA). If the sugar is ribose, the polymer is RNA; if the sugar is the ribose derivative deoxyribose, the polymer is DNA. There are several forms of DNA, for example, but not limited to plasmid DNA or genomic DNA. Similarly, RNA can be found in the form of messenger RNA (mRNA), transfer-RNA (t-RNA), catalytical RNA or structural RNA. DNA or RNA molecules can also be synthetically prepared for different functions. In one embodiment, the nucleic acid is DNA or RNA. In one embodiment, the nucleic acid is DNA. In one embodiment, the nucleic acid is RNA.

Without wanting to be bound by a mechanism, it is believed that the viscosity reducing effect of the liquid compositions according to the invention is based upon an interaction between the excipients and the nucleic acid residues or the sucrose/phosphate backbone of the nucleic acid. Because all nucleic acids are built from the same pool of nucleotides, the effects described herein are thus applicable to all types of nucleic acids. The liquid compositions according to the invention therefore have an advantageous effect on any type of nucleic acid irrespective of its sequence, size and structure.

The term “liquid composition comprising a nuceic acid” as used herein, refers to a liquid aqueous nucleic acid containing composition, preferably an aqueous nucleic acid containing solution. The composition can contain one or more nucleic acids in different concentrations. Usually the concentration of one nucleic acid in the liquid composition is above 1, 3, 5 or 10 mg/ml.

In a preferred embodiment, the nucleic acid contained in the composition according to the invention is a therapeutic nucleic acid.

The term “therapeutic nucleic acid” as used herein refers to any nucleic acid that is administered to a subject with the aim of treating or preventing a disease or medical condition. In particular, the subject may be a mammal or a human. Therapeutic proteins can be administered for different purposes, such as replacing a protein that is deficient or abnormal, augmenting an existing pathway, providing a novel function or activity, interfering with a molecule or organism and delivering other compounds or proteins, such as a radionuclide, cytotoxic drug, or effector proteins. Therapeutic nucleic acids encompass naked DNA/RNA or modified DNA/RNAs. Their sequence can be natural or engineered.

In a particularly preferred embodiment, the nucleic acid in the liquid compositions according to the invention is naked DNA, in particular a therapeutic plasmid.

In a further particularly preferred embodiment, the nucleic acid in the liquid compositions according to the invention is RNA, such as mRNA.

In one embodiment, the nucleic acid is a biosimilar. A “biosimilar” is herein defined as a biological medicine that is highly similar to another already approved biological medicine. In a preferred embodiment, the biosimilar is a RNA, such as mRNA.

In one embodiment, the liquid compositions according to the invention comprise more than one type of nucleic acid.

The term “excipient”, as used herein, refers to any compound at a suitable concentration which is known to reduce the viscosity of a composition comprising a nucleic acid by at least 5% compared to an identical composition not comprising the excipient.

The liquid compositions according to the invention comprise at least one excipient selected from the group consisting of
arginine (CAS-Registry Number 74-79-3)
phenylalanine (CAS-Registry Number 63-91-2)
ornithine (CAS-Registry Number 3184-13-2)
meglumine (CAS-Registry Number 6284-40-8)
benzenesulfonic acid (CAS-Registry Number 98-11-3)
pyridoxine (vitamin B6, CAS-Registry Number 65-23-6),
thiamine phosphoric acid ester (thiamine monophosphate, CAS-Registry Number 10023-48-0).

According to the invention, the excipients include salts or solvates of the excipients. Preferred salts in the context of the present invention are physiologically acceptable salts of the compounds according to the invention. Salts which are not themselves suitable for pharmaceutical uses but can be used, for example, for isolation, purification or storage of the compounds according to the invention are also included.

Physiologically acceptable salts of the compounds according to the invention include salts of conventional bases, such as, by way of example and preferably, alkali metal salts (e.g. sodium and potassium salts), alkaline earth metal salts (e.g. calcium and magnesium salts) and ammonium salts derived from ammonia or organic amines having 1 to 16 C atoms, such as, by way of example and preferably, ethylamine, diethylamine, triethylamine, N,N-diisopropylethylamine, monoethanolamine, diethanolamine, triethanolamine, dimethylaminoethanol, diethylaminoethanol, procaine, dicyclohexylamine, dibenzylamine, N-methylpiperidine, N-methylmorpholine, arginine, lysine and 1,2-ethylenediamine. Physiologically acceptable salts of the compounds according to the invention include salts of conventional acids, such as, by way of example and preferably, acetate, adipate, alginate, citrate, aspartate, benzoate, benzenesul-fonate, bisulfate, butyrate, camphorate, camphorsulfonate, carbonate, digluconate, glycerophosphate, hemisulfate, heptanoate, hexanoate, formate, fumarate, hydrochloride, hydrobromide, hydroiodide, 2-hydroxyethansulfonate (isethionate), lactate, maleate, mesitylenesulfonate, methanesulfonate, naphthyl-enesulfonate, nicotinate, 2-naphthalenesulfonate, oxalate, pamoate, pectinate, persulfate, 3-phenylproprionate, picrate, pivalate, propionate, succinate, sulfate, tartrate, trichloroacetate, trifluoroacetate, phosphate, glutamate, bicarbonate, para-toluenesulfonate, and undecanoate.

By way of example, physiologically acceptable salts can be salts of ornithine, e.g. ornithine monohydrochloride.

Solvates in the context of the invention are designated as those forms of the compounds according to the invention which form a complex in the solid or liquid state by coordination with solvent molecules. Hydrates are a specific form of solvates, in which the coordination takes place with water. Hydrates are preferred solvates in the context of the present invention.

According to the invention, the excipients include all enantionmers and stereoisomers and racemic mixtures of the excipients. By way of example, the excipient arginine includes L-arginine, D-arginine and racemic mixtures of L- and D-arginine.

The liquid compositions according to the invention comprise an amount of the excipient sufficient to reduce the viscosity of the composition and/or stabilize the protein. For example, the liquid compositions according to the invention may comprise about 5 mM to about 300 mM, about 5 mM to about 250 mM or about 5 mM to about 150 mM of the at least one excipient. In exemplary embodiments the concentration of the at least one excipient is 1, 5, 10, 12, 13, 15, 20, 25, 30, 35, 50, 75, 100, 105, 110, 115, 120, 125, 130, 135, 140, 145, 150, 155, 160, 165, 170, 175, 180, 185, 190, 195, 200, 210, 220, 250, or 300 mM or greater.

In one aspect of the invention these liquid compositions may further comprise additives that are used for purposes other than reducing viscosity, e.g. stabilization, solubilization or preservation.

In another aspect of the invention, the liquid compositions comprise more than one excipient. For example, the liquid compositions according to the invention may comprise two, three or four excipients, preferably they contain two excipients.

In the following, combinations of two excipients are mentioned that are advantageous for the liquid compositions comprising a nucleic acid, the lyophilized formulations of the liquid compositions comprising a nucleic acid, the method for reducing the viscosity of liquid compositions comprising a nucleic acid and the kit comprising a composition comprising a nucleic acid as described in the present invention.

One embodiment of the invention is a liquid composition, comprising a combination of two excipients selected from the group consisting of arginine, phenylalanine, ornithine, meglumine, benzenesulfonic acid, pyridoxine and thiamine phosphoric acid ester.

One embodiment of the invention is a liquid composition, comprising a combination of a first excipient selected from the group consisting of arginine, phenylalanine, ornithine and meglumine and a second excipient selected from the group consisting of benzenesulfonic acid, pyridoxine and thiamine phosphoric acid ester.

One embodiment of the invention is a liquid composition, comprising a combination of a first excipient selected from the group consisting of arginine, ornithine and meglumine and a second excipient selected from the group consisting of benzenesulfonic acid, pyridoxine and thiamine phosphoric acid ester.

Combinations of two excipients include: arginine and phenylalanine, arginine and ornithine, arginine and meglumine, arginine and benzenesulfonic acid, arginine and pyridoxine, arginine and thiamine phosphoric acid ester, phenylalanine and ornithine, phenylalanine and meglumine, phenylalanine and benzenesulfonic acid, phenylalanine and pyridoxine, phenylalanine and thiamine phosphoric acid ester, ornithine and meglumine, ornithine and benzenesulfonic acid, ornithine and pyridoxine, ornithine and thiamine phosphoric acid ester, meglumine and benzenesulfonic acid, meglumine and pyridoxine, meglumine and thiamine phosphoric acid ester, benzenesulfonic acid and pyridoxine, benzenesulfonic acid and thiamine phosphoric acid ester, pyridoxine and thiamine phosphoric acid ester.

Preferred combinations of two excipients include arginine and benzenesulfonic acid, arginine and pyridoxine, arginine and thiamine phosphoric acid ester, phenylalanine and benzenesulfonic acid, phenylalanine and pyridoxine, phenylalanine and thiamine phosphoric acid ester, ornithine and benzenesulfonic acid, ornithine and pyridoxine, ornithine and thiamine phosphoric acid ester, meglumine and benzenesulfonic acid, meglumine and pyridoxine, meglumine and thiamine phosphoric acid ester.

More preferred combinations of two excipients include arginine and benzenesulfonic acid, arginine and pyridoxine, arginine and thiamine phosphoric acid ester, ornithine and benzenesulfonic acid, ornithine and pyridoxine, ornithine and thiamine phosphoric acid ester, meglumine and benzenesulfonic acid, meglumine and pyridoxine, meglumine and thiamine phosphoric acid ester.

In one embodiment, arginine and benzenesulfonic acid are combined. In another embodiment, arginine and pyridoxine are combined. In another embodiment, arginine and thiamine phosphoric acid ester are combined. In another embodiment, phenylalanine and benzenesulfonic acid are combined. In another embodiment, phenylalanine and pyridoxine are combined. In another embodiment, phenylalanine and thiamine phosphoric acid ester are combined. In another embodiment, ornithine and benzenesulfonic acid are combined. In another embodiment, ornithine and pyridoxine are combined. In another embodiment, ornithine and thiamine phosphoric acid ester are combined. In another embodiment, meglumine and benzenesulfonic acid are combined. In another embodiment, meglumine and pyridoxine are combined. In another embodiment, meglumine and thiamine phosphoric acid ester are combined. In another embodiment, meglumine and pyridoxine are combined. In another embodiment, meglumine and thiamine phosphoric acid ester are combined. In another embodiment, meglumine and pyridoxine are combined. In another embodiment, meglumine and thiamine phosphoric acid ester are combined.

The liquid compositions according to the invention comprise an amount of a combination of two excipients as mentioned above, wherein the concentration of both excipients is sufficient to reduce the viscosity of the liquid composition and or to stabilize the nucleic acid. For example, the liquid compositions according to the invention may comprise about 5 mM to about 300 mM, about 5 mM to about 250 mM or about 5 mM to about 150 mM of each excipient. In exemplary embodiments the concentration of each excipient is 1, 5, 10, 12, 13, 15, 20, 25, 30, 35, 50, 75, 100, 105, 110, 115, 120, 125, 130, 135, 140, 145, 150, 155, 160, 165, 170, 175, 180, 185, 190, 195, 200, 210, 220, 250, or 300 mM or greater.

In one embodiment the liquid compositions comprise a combination of two excipients, wherein the molar concentration of the excipients can be identical or different. The molar ratio of the first and second excipient is between 1:100 and 100:1, preferably between 1:10 and 10:1, more preferably between 1:5 and 5:1, most preferably between 1:2 and 2:1. In a particular embodiment the molar concentration of the two excipients is identical.

The excipients, excipient combinations, concentrations and molar ratios of the excipients and excipient combinations, concentrations of the nucleic acid as disclosed above for the liquid composition comprising a nucleic acid and at least one excipient, shall apply to the same extend to all other embodiments of the invention, including the lyophilized formulation of the liquid composition, the method for reducing the viscosity of a liquid composition or the use of the method in a bioprocess.

In a further preferred embodiment of the liquid compositions, the method for reducing the viscosity or increasing stability, the excipients synergistically reduce the viscosity and/or increase stability in the liquid compositions comprising a nucleic acid.

According to the invention, a synergistical reduction of the viscosity is given if the viscosity reduction by a combination of two or more excipients is more than the expected sum of the viscosity reduction of each individual excipient. Preferably a synergistical reduction of the viscosity is given if the percentage viscosity reduction by a combination of two or more excipients is more than the expected sum of the percentage viscosity reduction of each individual excipient.

In one embodiment all combinations of two excipients mentioned in the present application result in a synergistic reduction of viscosity of a liquid composition comprising a nucleic acid.

In another aspect, the invention provides a lyophilized formulation of the liquid composition comprising a nucleic acid and at least one excipient selected from the group consisting of arginine, phenylalanine, ornithine, meglumine, benzenesulfonic acid, pyridoxine and thiamine phosphoric acid ester. Upon reconstitution with a suitable amount of diluent, the formulations exhibit reduced viscosity relative to control formulations with the otherwise same composition but not comprising the at least one excipient. Thus, the excipient is present at an amount effective to reduce viscosity upon reconstitution with diluent.

A lyophilized formulation according to the invention includes a nucleic acid and the at least one excipient according to the invention that has been dried and is present as particles in, for example, powder form. In the present context the expression “powder” refers to a collection of essentially dry particles, i.e. the moisture content being at least below about 10% by weight, 6% by weight, 4% by weight, or lower.

As defined herein, “viscosity” refers to the resistance of a substance (typically a liquid) to flow. Viscosity is related to the concept of shear force; it can be understood as the effect of different layers of the fluid exerting shearing force on each other, or on other surfaces, as they move against each other. There are several ways to express viscosity. The units of viscosity are Ns/m², known as Pascal-seconds (Pas). Viscosity can be “kinematic” or “absolute”. Kinematic viscosity is a measure of the rate at which momentum is transferred through a fluid. It is measured in Stokes (St). The kinematic viscosity is a measure of the resistive flow of a fluid under the influence of gravity. When two fluids of equal volume and differing viscosity are placed in identical capillary viscometers and allowed to flow by gravity, the more viscous fluid takes longer than the less viscous fluid to flow through the capillary. If, for example, one fluid takes 200 seconds(s) to complete its flow and another fluid takes 400 s, the second fluid is called twice as viscous as the first on a kinematic viscosity scale. The dimension of kinematic viscosity is length²/time. Commonly, kinematic viscosity is expressed in centiStokes (cSt). The SI unit of kinematic viscosity is mm²/s, which is equal to 1 cSt. The “absolute viscosity,” sometimes called “dynamic viscosity” or “simple viscosity,” is the product of kinematic viscosity and fluid density. Absolute viscosity is expressed in units of centipoise (cP). The SI unit of absolute viscosity is the milliPascal-second (mPas), where 1 cP=1 mPas.

Viscosity may be measured by using, for example, a viscometer at a given shear rate or multiple shear rates. An “extrapolated zero-shear” viscosity can be determined by creating a best fit line of the four highest-shear points on a plot of absolute viscosity versus shear rate, and linearly extrapolating viscosity back to zero-shear. Alternatively, for a Newtonian fluid, viscosity can be determined by averaging viscosity values at multiple shear rates. Viscosity can also be measured using a microfluidic viscometer at single or multiple shear rates (also called flow rates), wherein absolute viscosity is derived from a change in pressure as a liquid flows through a channel. Viscosity equals shear stress over shear rate. Viscosities measured with microfluidic viscometers can, in some embodiments, be directly compared to extrapolated zero-shear viscosities, for example those extrapolated from viscosities measured at multiple shear rates using a cone and plate viscometer. According to the invention, viscosity of liquid compositions is reduced when at least one of the methods described above show a stabilizing effect. Preferably, viscosity is measured at 20° C. using a microfluidic viscometer. More preferably the viscosity is measured using a RheoSense mVROC microfluidic viscometer at 20° C. Most preferably the viscosity is measured at 20° C. using a RheoSense mVROC microfluidic viscometer and using a 250 μl syringe, a shear rate of 1500 s−1 or 1000 s−1 and a volume of 60 to 80 μl.

The person ordinary skilled in the art is familiar with the viscosity measurement using a microfluidic viscometer. As microfluidic viscometer the RheoSense mVROC microfluidic viscometer (mVROC™ Technology), especially with the parameters descriped above can be used. Detailed specifications, methods and setting can be found in the 901003.5.1-mVROC_User's_Manual.

“Shear rate” herein refers to the rate of change of velocity at which one layer of fluid passes over an adjacent layer. The velocity gradient is the rate of change of velocity with distance from the plates. This simple case shows the uniform velocity gradient with shear rate (v1−v2)/h in units of (cm/sec)/(cm)=1/sec. Hence, shear rate units are reciprocal seconds or, in general, reciprocal time. For a microfluidic viscometer, change in pressure and flow rate are related to shear rate. “Shear rate” is to the speed with which a material is deformed. Formulations containing nucleic acids and excipients are typically measured at shear rates ranging from about 0.5 s⁻¹ to about 200 s⁻¹ when measured using a cone and plate viscometer and a spindle appropriately chosen by one skilled in the art to accurately measure viscosities in the viscosity range of the sample of interest (i.e., a sample of 20 cP is most accurately measured on a CPE40 spindle affixed to a DV2T viscometer (Brookfield)); greater than about 20 s⁻¹ to about 3,000 s⁻¹ when measured using a microfluidic viscometer.

For classical “Newtonian” fluids, as generally used herein, viscosity is essentially independent of shear rate. For “non-Newtonian fluids,” however, viscosity either decreases or increases with increasing shear rate, e.g., the fluids are “shear thinning” or “shear thickening”, respectively. In the case of concentrated (i.e., high-concentration) liquid compositions comprising a nucleic acid, this may manifest as pseudoplastic shear-thinning behavior, i.e., a decrease in viscosity with shear rate.

In one embodiment, the liquid compositions of the invention show a reduction of viscosity of at least 5%, 10%, 15%, 20%, 25%, 30%, 35%, 40%, 45%, 50%, 55%, 60%, 65%, 70% or 75% compared to an identical composition not comprising the at least one excipient.

In one embodiment, the liquid compositions of the invention show a reduction of viscosity of at least 5%, 10%, 15%, 20%, 25%, 30%, 35%, 40%, 45%, 50%, 55%, 60%, 65%, 70% or 75% compared to an identical composition not comprising the combination of two excipients.

The liquid compositions according to the invention may additionally comprise pharmaceutically acceptable diluents, solvents, carriers, adhesives, binders, preservatives, solubilizers, surfactants, penetration enhancers, stabilizers, emulsifiers or bioavailability enhancers. The skilled person knows how to choose suitable additives for liquid compositions comprising a nucleic acid that are safe and well tolerated.

The invention further provides a liquid composition according to the invention whereas the nucleic acid has a molecular weight of 100 base pairs or more, preferably from 50 to 50000 base pairs.

In a preferred embodiment, the nucleic acid concentration in the liquid compositions according to the invention is at least 1 mg/ml, at least 3 mg/ml, at least 5 mg/ml, preferably at least 7.5 mg/ml and more preferably at least 10 mg/ml. In another preferred embodiment, the nucleic acid concentration is between 1 mg/ml and 50 mg/ml or between 3 mg/ml and 50 mg/ml or between 3 mg/ml and 30 mg/ml or between 3 mg/ml and 20 mg/ml or between 1 mg/ml and 10 mg/ml.

The invention further provides a liquid composition according to the invention further comprising a buffer at a concentration of 10 mM to 50 mM. The buffer can be a suitable tris(hydroxymethyl)aminomethane salt and provide a pH of around 7. Said buffer can be supplemented with other components such as ethylenediaminetetraacetic acid (TE-Buffer); ethylenediaminetetraacetic acid and sodium chloride (TSE-Buffer), or ethylenediaminetetraacetic acid and acetic acid (TAE-Buffer). Instead of acetic acid other acids such as boric acid or phosphoric acid could be used.

The invention further provides a liquid composition according to the invention whereas the viscosity is between 1 mPas and 60 mPas, preferably between 1 mPas and 50 mPas, more preferably between 1 mPas and 30 mPas, most preferably between 1 mPas and 20 mPas. Preferably the viscosity is measured at 20° C., using a microfluidic viscometer. More preferably the viscosity is measured using a RheoSense mVROC microfluidic viscometer at 20° C. Most preferably the viscosity is measured using a RheoSense mVROC microfluidic viscometer at 20° C., using a 250 μl syringe, a shear rate of 1500 s−1 or 1000 s−1 and a volume of 60-80 μl

The invention is also directed to a kit comprising a lyophilized formulation according to the invention, optionally in a container, and instructions for its reconstitution and administration, optionally with a vial of sterile diluent, and optionally with a syringe or other administration device. Exemplary containers include vials, tubes, bottles, single or multi-chambered pre-filled syringes, or cartridges, but also a 96-well plate comprising ready-to-use freeze-dried or spray-dried formulations sitting in the wells. Exemplary administration devices include syringes with or without needles, infusion pumps, jet injectors, pen devices, transdermal injectors, or other needle-free injectors.

The invention is also directed to methods for reducing the viscosity of a liquid composition comprising a nucleic acid for the purpose of the manufacture of all aforementioned liquid compositions. All embodiments regarding the combinations and concentrations of excipients, nucleic acids, concentrations and molecular weight of nucleic acids, pH, buffer and buffer concentrations as mentioned for the liquid composition above are also applicable for the methods for reducing the viscosity of a composition comprising a nucleic acid.

The invention further provides a method for reducing the viscosity of a liquid composition comprising a nucleic acid, comprising a step of adding at least one excipient selected from the group consisting of arginine, phenylalanine, ornithine, meglumine, benzenesulfonic acid, pyridoxine and thiamine phosphoric acid ester or salts or solvates thereof to the liquid nucleic acid composition.

The invention further provides a method for reducing the viscosity of a liquid composition comprising a nucleic acid, comprising a step of adding a combination of two excipients selected from the group consisting of arginine, phenylalanine, ornithine, meglumine, benzenesulfonic acid, pyridoxine and thiamine phosphoric acid ester or salts or solvates thereof, is added to the liquid nucleic acid composition.

The invention further provides a method for reducing the viscosity of a liquid composition comprising a nucleic acid, comprising a step of adding a combination of a first excipient selected from the group consisting of arginine, phenylalanine, ornithine and meglumine and a second excipient selected from the group consisting of benzenesulfonic acid, pyridoxine and thiamine phosphoric acid ester.

All embodiments mentioned above for the liquid composition comprising the nucleic acid according to the invention also apply for the method for reducing the viscosity of a liquid composition comprising a nucleic acid. This includes all embodiments regarding the nature and concentration of the nucleic acid, combinations of excipients, concentrations and ratios of the excipients, additional ingredients of the liquid composition, viscosity reduction of the liquid composition or stability of the nucleic acids.

Likewise, the invention is directed to methods for increasing the stability of a liquid composition comprising a nucleic acid, comprising the steps as mentioned for the method of reducing the viscosity of a liquid composition comprising a nucleic acid.

The invention further provides a method of preventing self-association of a nucleic acid in a liquid composition comprising the nucleic acid, comprising the steps as mentioned for the method of reducing the viscosity of a liquid composition comprising a nucleic acid.

In the methods according to the invention, the excipients may be added in any way known to the artisan to the composition comprising a nucleic acid. When more than one excipient is added, the excipients may be pre-mixed and subsequently added to the liquid composition comprising the nucleic acid. Likewise, the excipients may be added separately to the liquid composition comprising the nucleic acid.

In a preferred embodiment of the invention the liquid composition is a pharmaceutical composition. The invention is also directed to a pharmaceutical composition as described above comprising a therapeutic nucleic acid for the treatment of disease.

In particular pharmaceutical liquid compositions as described above comprising a therapeutic nucleic acid are suitable for the treatment of cancer, rheumatoid arthritis, morbus crohn, colitis ulcerose, ankylosing spondylitis, psoriasis-arthritis, psoriasis, hypercholesterolemia, mixed dyslipidemia, homozygous familial hypercholesterolemia, myocardial infaction, peripheral arterial disease or immune deficiency disorders.

The invention is also directed to methods of treatment, wherein the treatment comprises administration of a pharmaceutical composition as described above comprising a therapeutic nucleic acid.

In one aspect, the method of treatment is a method of treating cancer. That is, the liquid compositions according to the invention are useful for the treatment of cancer, rheumatoid arthritis, morbus crohn, colitis ulcerose, ankylosing spondylitis, psoriasis-arthritis, psoriasis, hypercholesterolemia, mixed dyslipidemia, homozygous familial hypercholesterolemia, myocardial infaction, peripheral arterial disease or immune deficiency disorders.

Furthermore, it has been found that the excipients and excipient combinations as mentioned above are beneficial in the bioprocess.

In bioprocesses as meant here, the addition of these excipients, which are found to serve as viscosity-reducing additives, lead to an improved process economy, in that on the one hand the yield of intact nucleic acid can be improved, and on the other hand the duration of the process can be reduced.

In technical approaches, where a solution is passed through a filter or medium, like a gel bed, a packed or fluidized bed of porous or non-porous particles, a membare, or monolith by a force applied by back-end pressure, e.g. during chromatographic purifications, the disclosed excipients have beneficial effects. Back pressure of chromatography materials can drastically increase during elution of nucleic acids, posing potential risk of process failures in case of complete blockage, an event which can occurs in particular with high capacity chromatography materials operated at high loading. While said filtration steps are mainly used in the downstream process, excipients can also be beneficial in the upstream process. When nucleic acid concentrations elevate to levels where they cause viscosity, with the described negative effects of pressure limitations and shear forces when the solution is passed through a tubing or a filter to remove cellular material and debris the presented invention will obviously have beneficial effects. To measure process efficiency in tangential flow filtration, where in contrast to previously used method the majority of the field flow travels tangentially across the surface of the filter, rather than passing through the filter, a laboratory scale TFF system was used. While the filtration principle is different than in the methods described previously, also here the filtration efficiency depends on the resistance of the membrane, which also here remains constant, and the solution viscosity, which is modified by the invention. Filtration methods are typical unit operation used to exchange a formulation buffer or to bring the concentration of a biomolecule to the desired level. The stirred cells used herein are representation of dead-end filters, where a feed is passed through a filtering material that withholds larger molecules on top of the material releasing the filtrate on the other end of the device. While in TFF mode concentration polarization and clogging of the membrane surface is reduced by introducing a sufficient cross-flow velocity of feed solution, stirred cells use a stirrer to create turbulence of feed solution over the membrane surface.

A frequent method to exchange buffers and to concentrate nucleic acids is tangential flow filtration, where in contrast to previously used methods the majority of the field flow travels tangentially across the surface of the filter, rather than passing through the filter. Like when stirred cells are used in tangential flow filtration the large molecules are separated from smaller molecules by passing said smaller molecules through a suitable filter material. In contrast to stirred cells, which represent one form of dead end filtration, in tangential flow filtration the flow geometry of the feed is different to avoid the formation of a filter cake and allowing for a continuous process. When stirred cells are used the formation of a filter cake is likewise prevented by the use of a stirring device. Therefore, the stirred cells closely resemble a tangential flow filtration device in spite of the differences in filter geometry. The efficiency of both methods is critically depending on the membrane resistance. Also, a high viscosity is known to reduce the flux rate that can be used and therefore increasing processing time resulting in higher production costs. It is therefore expected that a reduced viscosity allows for a more efficient filtration process while shear forces remain low yielding to a higher nucleic acid concentration in the filtrate. This is highlighted by the work of Hung et al. who state: “During production of concentrated monoclonal antibody formulations by tangential flow ultrafiltration (TFF), high viscosities and aggregation often cause extensive membrane fouling, flux decay and low product yields” (Journal of Membrane Science Volume 508, 15 Jun. 2016, Pages 113-126)

Excipients selected from the group consisting of arginine, phenylalanine, ornithine, meglumine, benzenesulfonic acid, pyridoxine and thiamine phosphoric acid ester or salts or solvates thereof are suitable to improve bioprocess economics as described before. Especially combinations in various ratios depending on the liquid compositions comprising a nucleic acid improve bioprocess economics as described.

Therefore, another aspect of the present invention is to provide a method for reducing the viscosity of a composition comprising a nucleic acid in a bioprocess, comprising the step of combining the composition comprising a nucleic acid with at least one excipient selected from the group consisting of arginine, phenylalanine, ornithine, meglumine, benzenesulfonic acid, pyridoxine and thiamine phosphoric acid ester or salts or solvates thereof.

In another aspect, the composition comprising a nucleic acid is combined with a combination of two excipients selected from the group consisting of arginine, phenylalanine, ornithine, meglumine, benzenesulfonic acid, pyridoxine and thiamine phosphoric acid ester or salts or solvates thereof.

In another aspect, the composition comprising a nucleic acid is combined with a combination of a first excipient selected from the group consisting of arginine, phenylalanine, ornithine and meglumine and a second excipient selected from the group consisting of benzenesulfonic acid, pyridoxine and thiamine phosphoric acid ester or salts or solvates thereof.

According to the present invention all parameters mentioned above; e.g. combinations of the excipients, concentrations of the excipients, concentrations the nucleic acid, ratios of the excipients, further components of the composition, pH values, viscosity reductions, specifications of the nucleic acid also apply to the method for reducing the viscosity of a composition comprising a nucleic acid in a bioprocess.

Another aspect of the present invention is to provide a method for reducing the viscosity of a composition comprising a nucleic acid in a bioprocess as mentioned above, wherein the permeate flux of the composition comprising a nucleic acid in a filtration step is increased compared to an identical composition comprising a nucleic acid not comprising at least one excipient selected from the group consisting of arginine, phenylalanine, ornithine, meglumine, benzenesulfonic acid, pyridoxine and thiamine phosphoric acid ester or compared to an identical composition comprising a nucleic acid not comprising a combination of two excipients selected from the group consisting of arginine, phenylalanine, ornithine, meglumine, benzenesulfonic acid, pyridoxine and thiamine phosphoric acid ester or compared to an identical composition comprising a nucleic acid not comprising a combination of a first excipient selected from the group consisting of arginine, phenylalanine, ornithine and meglumine and a second excipient selected from the group consisting of benzenesulfonic acid, pyridoxine and thiamine phosphoric acid ester.

Another aspect of the present invention is the use of the method in a bioprocess as mentioned above, wherein the permeate flux of the composition comprising a nucleic acid in a filtration step is increased compared to an identical composition comprising a nucleic acid not comprising at least one excipient selected from the group consisting of arginine, phenylalanine, ornithine, meglumine, benzenesulfonic acid, pyridoxine and thiamine phosphoric acid ester or compared to an identical composition comprising a nucleic acid not comprising a combination of two excipients selected from the group consisting of arginine, phenylalanine, ornithine, meglumine, benzenesulfonic acid, pyridoxine and thiamine phosphoric acid ester or compared to an identical composition comprising a nucleic acid not comprising a combination of a first excipient selected from the group consisting of arginine, phenylalanine, ornithine and meglumine and a second excipient selected from the group consisting of benzenesulfonic acid, pyridoxine and thiamine phosphoric acid ester.

Increase of permeate flux means a percentage increase of at least 2%, preferably at least 5%, more preferably at least 10%, most preferred 10% to 100%.

Another aspect of the present invention is to provide a method for reducing the viscosity of a composition comprising a nucleic acid in a bioprocess as mentioned above, wherein the nucleic acid recovery after buffer exchange and volume reduction in filters is increased compared to an identical composition comprising a nucleic acid not comprising at least one excipient selected from the group consisting of arginine, phenylalanine, ornithine, meglumine, benzenesulfonic acid, pyridoxine and thiamine phosphoric acid ester or compared to an identical composition comprising a nucleic acid not comprising a combination of two excipients selected from the group consisting of arginine, phenylalanine, ornithine, meglumine, benzenesulfonic acid, pyridoxine and thiamine phosphoric acid ester or compared to an identical composition comprising a nucleic acid not comprising a combination of a first excipient selected from the group consisting of arginine, phenylalanine, ornithine and meglumine and a second excipient selected from the group consisting of benzenesulfonic acid, pyridoxine and thiamine phosphoric acid ester.

Increase of nucleic acid recovery after buffer exchange and volume reduction in filters means a percentage increase of nucleic acid recovery of at least 1%, preferably at least 2%, more preferably at least 5%, most preferred 5% to 20%.

Another aspect of the present invention is to provide a method for reducing the viscosity of a composition comprising a nucleic acid in a bioprocess as mentioned above, wherein the process time for a filtration step, preferably a filtration step wherein the nucleic acid is concentrated, is reduced compared to an identical composition comprising a nucleic acid not comprising at least one excipient selected from the group consisting of arginine, phenylalanine, ornithine, meglumine, benzenesulfonic acid, pyridoxine and thiamine phosphoric acid ester or compared to an identical composition comprising a nucleic acid not comprising a combination of two excipients selected from the group consisting of arginine, phenylalanine, ornithine, meglumine, benzenesulfonic acid, pyridoxine and thiamine phosphoric acid ester or compared to an identical composition comprising a nucleic acid not comprising a combination of a first excipient selected from the group consisting of arginine, phenylalanine, ornithine and meglumine and a second excipient selected from the group consisting of benzenesulfonic acid, pyridoxine and thiamine phosphoric acid ester.

Reduction of process time for a filtration step means a percentage reduction of at least 5%, preferably at least 10%, more preferably at least 25%, most preferred 25% to 100%.

In a particular embodiment of the invention, the filtration step is a tangential flow filtration (TFF).

The term “bioprocess” refers to therapeutic cell manufacturing processes, which can be separated into upstream processes and downstream processes. The upstream process is defined as the entire process prior to separating nucleic acid from cellular compounds. The upstream process comprises early cell isolation and cultivation, to cell banking and culture expansion of the cells until final harvest. The downstream part of a bioprocess refers to the part where the target nucleic acid is purified from the feed of the upstream and is processed to meet purity and quality requirements. Some type of cells need to be disrupted when entering the downstream process. Yet other cells may secrete the target nucleic acid into the media and need to be removed via filtration. Further downstream processing is usually divided into the main sections: a purification section and a polishing section. A bioprocess can be a batch process or a semi-continuous or a continuous process.

The term “permeate flux” refers to the volume passing through a defined filter within a certain period of time, typically on the order of minutes.

The term “filtration step” refers to a process step where a liquid is passed through a material with a defined pore size allowing for the separation of materials based on their size. For some filters the pore size is defined in nanometers. Yet for other filters, the pore size is not directly defined, but the weight of a molecule to be withheld is given. Filtering materials can be placed in a way that they block the cross-section of the filtration device (dead-end filtration). Yet filtering materials can be placed in a way that the solution to be filtered is tangentially flowing across the surface of said material, e.g. tangential flow filtration. The filtering material can be a membrane, a glass filter, a metallic filter or a resin. The resin can be held in a chromatography column. The resin can be a cationic or anion exchange resin, an affinity resin, like a Protein A or glutathione resin, or a hydrophobic or hydrophilic resin.

The term “nucleic acid recovery” after buffer exchange and volume reduction refers to the fraction of nucleic acid to be retrieved after a process step.

The term “tangential flow filtration” or “TFF” refers to a method of filtration where a solution passes over a defined filter tangentially. Substances smaller than the filter pores are forced out of the solution through the filter by the pressure resulting from solution flow rate, viscosity, temperature and other factors.

### EXAMPLES

### 1. Viscosity Reducing Effect of Excipients on Desoxyribonucleic Acid in TE-Buffer, pH 7.4

### Buffer Preparation

A concentrated nuclease-free TE-Buffer was diluted by addition of nuclease-free water to result in a 1×TE-Buffer. pH was adjusted to pH 7.4 using HCl and NaOH if necessary.

### Sample Preparation

Excipient solutions of 150 mM Ornithine (Orn), Arginine (Arg), Meglumine (Meg), Benzenesulfonic acid (BSacid), Pyridoxin (Pyr) and Thiamine monophosphate (TMP) were prepared in TE-Buffer pH 7.4, respectively. Likewise a excipient solution of 125 mM Phenylalanine (Phe) was prepared in TE-Buffer, pH 7.4. The pH was adjusted using HCl or NaOH, if necessary.

A concentrated pDNA solution containing the desired excipients was prepared using centrifugal filters (Amicon, 30 kDA MWCO) to exchange the original buffer with a buffer containing the relevant excipients and to reduce the volume of the solution. The pDNA was subsequently diluted to 8760 μg/mL and 7000 μg/mL, respectively.

### DNA Concentration Measurements

DNA Concentration was determined using absorption spectroscopy applying Lambert-Beer's-Law. For absorption spectroscopy the absorbance at 260 nm was measured at 260 nm using a NanoDrop™ OneC (Thermo Fisher Scientific). Layer thickness was adjusted by the device to yield the optimal resolution for the sample's concentration.

### Viscosity Measurements

The mVROC™ Technology (Rheo Sense, San Ramon, California USA) was used for viscosity measurements. Measurements were performed using a 250 μl syringe and a shear rate of 1500 s⁻¹. A volume of 80 μl was used. All samples were measured as triplicates.

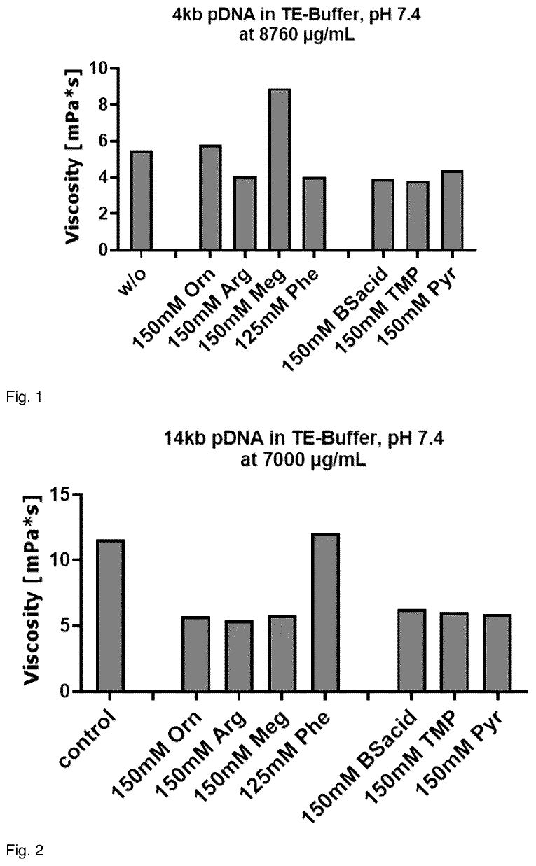

FIGS. 1 and 2 show the viscosity of the 4 kb pDNA and 14 kb pDNA solutions without (control) and with the different excipients.

### 2. Viscosity Reducing Effect of Excipient Combinations on Desoxyribonucleic Acid in TE-Buffer, pH 7.4

### Buffer Preparation

See Example 1.

### Sample Preparation

Excipient solutions of 75 mM Ornithine, Arginine, Phenylalanine or Meglumine were prepared in TE-Buffer pH 7.4 and supplemented with 75 mM Benzenesulfonic acid, Pyridoxin or Thiamine monophosphate, respectively. The pH was adjusted using HCl or NaOH, if necessary.

A concentrated pDNA solution containing the desired excipients was prepared using centrifugal filters (Amicon, 30 kDA MWCO) to exchange the original buffer with a buffer containing the relevant excipients and to reduce the volume of the solution. The pDNA was subsequently diluted to 9170 μg/mL and 7000 μg/mL, respectively.

### DNA Concentration Measurements

DNA Concentration was determined using absorption spectroscopy applying Lambert-Beer's-Law. For absorption spectroscopy the absorbance at 260 nm was measured at 260 nm using a NanoDrop™ OneC (Thermo Fisher Scientific).

Layer thickness was adjusted by the device to yield the optimal resolution for the sample's concentration.

### Viscosity Measurements

The mVROC™ Technology (Rheo Sense, San Ramon, California USA) was used for viscosity measurements. Measurements were performed using a 250 μl syringe and a shear rate of 1500 s−1. A volume of 80 μl was used. All samples were measured as triplicates.

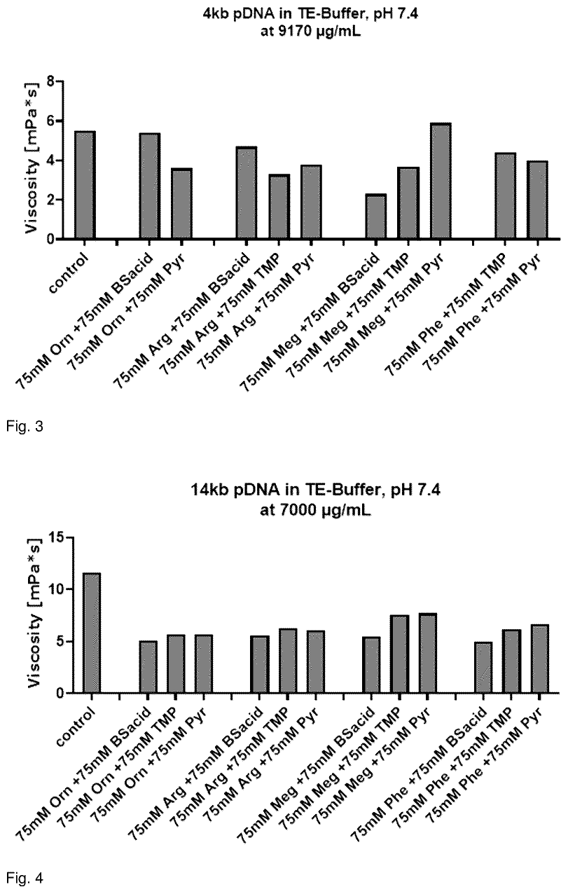

FIGS. 3 and 4 show the viscosity of the 4 kb pDNA and 14 kb pDNA solutions without (control) and with the different excipients.

### 3. Viscosity Reducing Effect of Excipients on Ribonucleic Acid in TE-Buffer, pH 7.0

### Buffer Preparation

A concentrated nuclease-free TE-Buffer was diluted by addition of nuclease-free water to result in a 1×TE-Buffer. pH was adjusted to pH 7.0 using HCl and NaOH if necessary.

### Sample Preparation

Excipient solutions of 150 mM Ornithine, Arginine, Benzenesulfonic acid, Pyridoxin, Thiamine monophosphate were prepared in TE-Buffer pH 7.0, respectively. Likewise a excipient solution of 125 mM Phenylalanine was prepared in TE-Buffer, pH 7.4. The pH was adjusted using HCl or NaOH, if necessary.

A concentrated mRNA solution containing the desired excipients was prepared using centrifugal filters (Amicon, 30 kDA MWCO) to exchange the original buffer with a buffer containing the relevant excipients and to reduce the volume of the solution. The mRNA was subsequently diluted to 8800 μg/mL, 5680 μg/mL and 7530 μg/mL, respectively.

mRNA Concentration Measurements

mRNA Concentration was determined using fluorescence spectroscopy. For fluorencence spectroscopy an assay (Quant-IT™ RNA XR Assay Kit, Thermo Fisher Scientific) was utilized. The fluorophores were excitated at 644 nm and fluorescence was measured at 673 nm using a Spark® Multimode Platereader (Tecan). The assay was performed and interpreted according to the manufacturer's instructions.

### Viscosity Measurements

The mVROC™ Technology (Rheo Sense, San Ramon, California USA) was used for viscosity measurements. Measurements were performed using a 250 μl syringe and a shear rate of 1500 s−1. A volume of 80 μl was used. All samples were measured as triplicates.

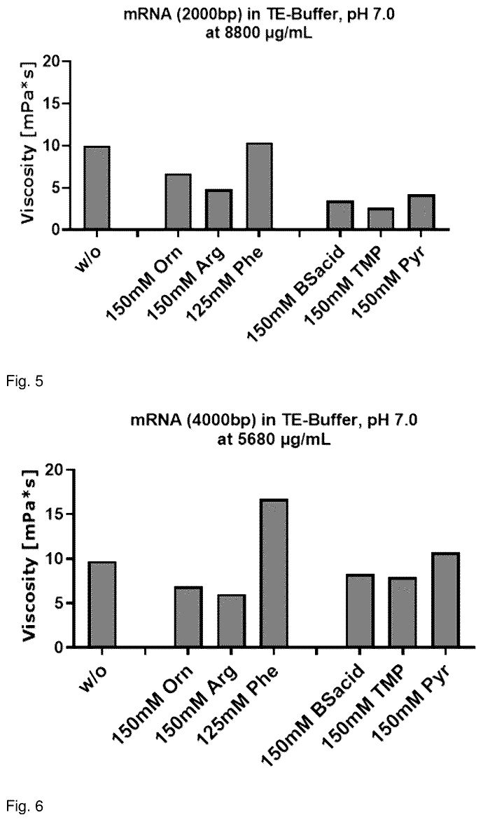

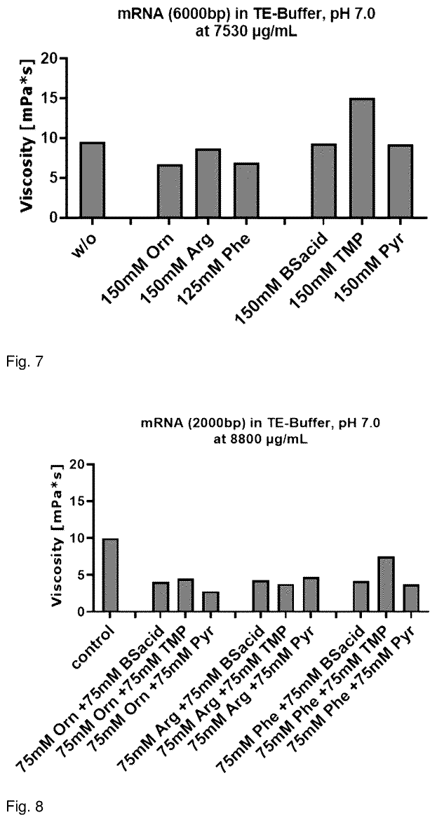

FIG. 5 to 7 show the viscosity of the 2000 bp, 4000 bp and 6000 bp mRNA solutions without (w/o) and with the different excipients.

### 4. Viscosity Reducing Effect of Excipient Combinations on Ribonucleic Acid in TE-Buffer, pH 7.0

### Buffer Preparation

A concentrated nuclease-free TE-Buffer was diluted by addition of nuclease-free water to result in a 1×TE-Buffer. pH was adjusted to pH 7.0 using HCl and NaOH if necessary.

### Sample Preparation

Excipient solutions of 75 mM Ornithine, Arginine, Phenylalanine were prepared in TE-Buffer pH 7.0 and supplemented with 75 mM Benzenesulfonic acid, Pyridoxin or Thiamine monophosphate, respectively. The pH was adjusted using HCl or NaOH, if necessary.

A concentrated mRNA solution containing the desired excipients was prepared using centrifugal filters (Amicon, 30 kDA MWCO) to exchange the original buffer with a buffer containing the relevant excipients and to reduce the volume of the solution. The mRNA was subsequently diluted to 8800 μg/mL, 5680 μg/mL and 7530 μg/mL, respectively.

mRNA Concentration Measurements

mRNA Concentration was determined using fluorescence spectroscopy. For fluorencence spectroscopy an assay (Quant-IT™ RNA XR Assay Kit, Thermo Fisher Scientific) was utilized. The fluorophores were excitated at 644 nm and fluorescence was measured at 673 nm using a Spark® Multimode Platereader (Tecan). The assay was performed and interpreted according to the manufacturer's instructions.

### Viscosity Measurements

The mVROC™ Technology (Rheo Sense, San Ramon, California USA) was used for viscosity measurements.

Measurements were performed using a 250 μl syringe and a shear rate of 1500 s−1. A volume of 80 μl was used. All samples were measured as triplicates.

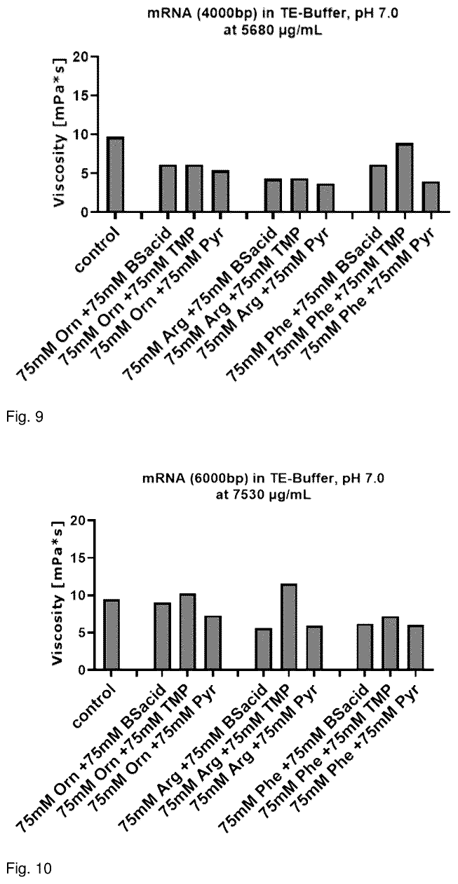

FIG. 8 to 10 show the viscosity of the 2000 bp, 4000 bp and 6000 bp mRNA solutions without (control) and with the different excipients.

1. A liquid composition comprising a nucleic acid and at least one excipient selected from the group consisting of arginine, phenylalanine, ornithine, meglumine, benzenesulfonic acid, pyridoxine and thiamine phosphoric acid ester.

2. A liquid composition according to claim 1, comprising a combination of two excipients selected from the group consisting of arginine, phenylalanine, ornithine, meglumine, benzenesulfonic acid, pyridoxine and thiamine phosphoric acid ester.

3. A liquid composition according to claim 1, comprising a combination of a first excipient selected from the group consisting of arginine, phenylalanine, ornithine and meglumine and a second excipient selected from the group consisting of benzenesulfonic acid, pyridoxine and thiamine phosphoric acid ester.

4. A liquid composition according to claim 1 having a reduced viscosity compared to an identical composition not comprising the at least one excipient.

5. A liquid composition according to claim 1, wherein the concentration of the nucleic acid is between 1 and 50 mg/ml.

6. A liquid composition according to claim 1, wherein the concentration of the at least one excipient is between 5 mM and 300 mM each.

7. A liquid composition according to claim 1, wherein the liquid composition has a viscosity between 1 mPas and 60 mPas at 20° C. as measured using a microfluidic viscometer.

8. A liquid composition according to claim 1, wherein the nucleic acid has a size of 100 base pairs or more.

9. A liquid composition according to claim 1, wherein the nucleic acid is DNA or RNA.

10. A lyophilized formulation of the liquid composition according to claim 1.

11. A method for reducing the viscosity of a liquid composition comprising a nucleic acid, comprising a step of adding at least one excipient selected from the group consisting of arginine, phenylalanine, ornithine, meglumine, benzenesulfonic acid, pyridoxine and thiamine phosphoric acid ester or salts or solvates thereof to a liquid nucleic acid composition.

12. A method according to claim 11, wherein a combination of two excipients selected from the group consisting of arginine, phenylalanine, ornithine, meglumine, benzenesulfonic acid, pyridoxine and thiamine phosphoric acid ester or salts or solvates thereof, is added to the liquid nucleic acid composition.

13. A method according to claim 11, wherein a combination of a first excipient selected from the group consisting of arginine, phenylalanine, ornithine and meglumine or salts or solvates thereof and a second excipient selected from the group consisting of benzenesulfonic acid, pyridoxine and thiamine phosphoric acid ester or salts or solvates thereof, is added to the liquid nucleic acid composition.

14. A method according to claim 11, wherein the nucleic acid is DNA or RNA.

15. A method for performing a bioprocess method, comprising processing of the liquid composition according to claim 1.
